(19) United States
(12) Patent Application Publication
(10) Pub. No.: US 2011/0071100 A1
(43) Pub. Date: Mar. 24, 2011

(54) SULFOBETAINES FOR CANCER, OBESITY, MACULAR DEGENERATION, NEURODEGENERATIVE DISEASES

(75) Inventors: Shmuel A. Ben-Sasson, Jerusalem (IL); Arie Dagan, Jerusalem (IL)

(73) Assignee: TILTAN PHARMA LTD., Jerusalem (IL)

(21) Appl. No.: 12/990,971

(22) PCT Filed: May 5, 2009
(86) PCT No.: PCT/IL2009/000465

§ 371 Date: Nov. 4, 2010

Related U.S. Application Data
(60) Provisional application No. 61/050,417, filed on May 5, 2008.

(51) Int. Cl. A61K 31/575; C07C 235/26; A61K 31/164; A61K 31/675; A61K 31/7068; A61P 35/00; A61P 29/00

(52) U.S. Cl. 514/49; 514/182; 514/171; 540/120; 514/625; 514/110

(57) ABSTRACT

The subject of the invention provides a pharmaceutical composition comprising a sulfobetaine, a sulfobetaine for therapy, uses thereof and methods of treating cancer obesity, age related macular degeneration or neurodegenerative diseases comprising administering a composition comprising a sulfobetaine.

### FIELD OF THE INVENTION

The subject invention relates to the field of sulfobetaines for therapy.

### BACKGROUND OF THE INVENTION

Sulfobetaines (SBs) are zwitterionic surfactants containing an ammonium cation, a sulfonate anion and a hydrophobic tail. SBs are used as mild detergents for protein solubilization and for basic study of micelle behavior in aqueous solutions, under various conditions (e.g. Graciani M et al. Langmuir 21: 7161-9 (2005)).

SBs, when tested in a comparative cancer therapy study, were found to have no therapeutic effect (Ip C. and Ganther H E. Carcinogenesis 13: 1167-70 (1992)).

SBs, when tested as an inhibitor of squalene synthase, were again found to have no therapeutic value (Spencer T A. et al. J. Org. Chem. 64: 807-18 (1999))

### SUMMARY OF THE INVENTION

The subject invention now provides sulfobetaines with a hydrophobic tail which do have therapeutic effects for a variety of diseases.

The present invention provides a pharmaceutical composition comprising a sulfobetaine of general formula (I)

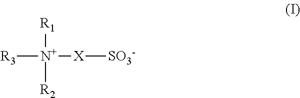

wherein
   X is —(CH₂)ₙ—, wherein n is 1-4, optionally substituted by at least one group selected from —(CH₂)ₚCH₃, —(CH₂)ₚOH, —(CH₂)ₚNH₂, and —(CH₂)ₚSH wherein p is 0-3;
   R₁ and R₂ independently of each other are each selected from a group consisting of H and —(CH₂)ₙCH₃ wherein m is 0-3; or
   R₁ and R₂ form together with the nitrogen atom a 5-8 membered hetero-aliphatic or hetero-aromatic ring;
   R₃ is a straight or branched C₁₄-C₂₅ alkyl, straight or branched C₁₄-C₂₅ alkenyl, or straight or branched C₁₄-C₂₅ alkynyl, each optionally substituted with at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkylamino, dialkylamino, carboxyl, or thio; and each optionally interrupted by at least one group selected from —C(═O)—NH—, —C(═S)—NH—, —C(═O)—SH—, —C(═O)—O—, —NH—C(═O)NH— and —NH—C(═S)NH—;
   or R₃ is a group having a formula (VIII)

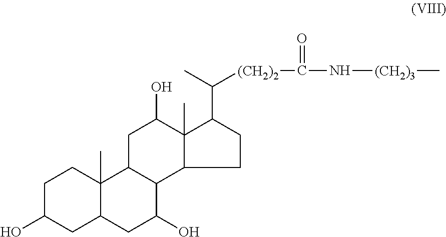

or enantiomers or diastereomers thereof; and a pharmaceutically acceptable carrier.

In a further aspect of the invention there is provided a sulfobetaine compound of formula (I)

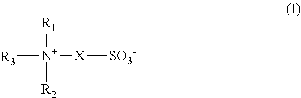

wherein X, R₁, R₂, R₃ are as defined above; or enantiomers thereof or diastereomers thereof and a pharmaceutically acceptable carrier, for therapy.

The invention further provides a use of a sulfobetaine of formula (I)

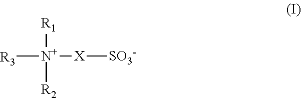

wherein X, R₁, R₂, R₃ are as defined above; or enantiomers thereof or diastereomers thereof and a pharmaceutically acceptable carrier for the manufacture of a medicament.

In another aspect the invention provides a method of treating cancer comprising administering a pharmaceutical composition of the invention. In another aspect the invention provides a method of treating obesity comprising administering a pharmaceutical composition of the invention. In another aspect the invention provides a method of treating age related macular degeneration comprising administering a pharmaceutical composition of the invention. The invention further provides a method of treating a neurodegenerative disease comprising administering a pharmaceutical composition of the invention.

The invention further provides a method of treating cancer in a subject in need thereof comprising administering a pharmaceutically effective amount of a compound of formula (II) to the subject

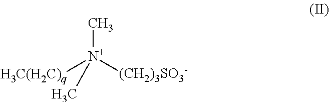

wherein q=14-20.

The invention additionally provides a method of treating cancer in a subject in need thereof comprising administering a pharmaceutically effective amount of a compound of formula (XI) to the subject

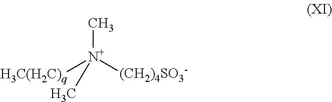

wherein q=14-20.

In a further aspect the invention encompasses a method of treating cancer in a subject in need thereof comprising administering a pharmaceutically effective amount of a compound of formula (XVI) to the subject

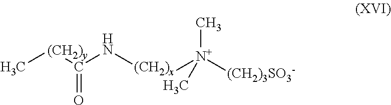

wherein x=1-4 and y=10-24.

In yet a further aspect the invention encompasses a method of treating cancer in a subject in need thereof comprising administering a pharmaceutically effective amount of a compound of formula (XVII) to the subject

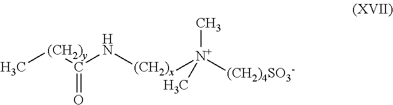

wherein x=1-4 and y=10-24.

The subject invention further provides, a method of treating cancer in a subject in need thereof comprising administering a pharmaceutically effective amount of a compound of formula (X) to the subject

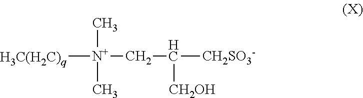

wherein q=14-20.

In another one of its aspects the invention provides a method of treating cancer in a subject in need thereof comprising administering a pharmaceutically effective amount of a compound of formula (III):

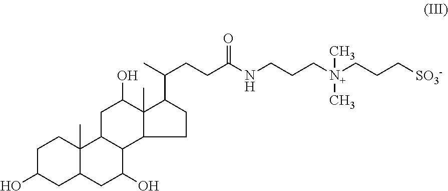

to the subject.

Furthermore, the invention provides a method of treating cancer in a subject in need thereof comprising administering a pharmaceutically effective amount of a compound of formula (IX):

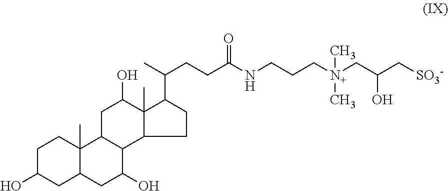

to the subject.

In another aspect the invention provides a method of treating obesity in a subject in need thereof comprising administering a pharmaceutically effective amount of a compound of formula (II) to the subject

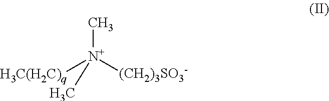

wherein q=14-20.

In a further aspect the invention provides a method of treating obesity in a subject in need thereof comprising administering a pharmaceutically effective amount of a compound of formula (XI) to the subject

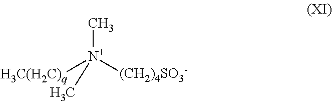

wherein q=14-20.

In a further aspect the invention encompasses a method of treating obesity in a subject in need thereof comprising administering a pharmaceutically effective amount of a compound of formula (XVI) to the subject

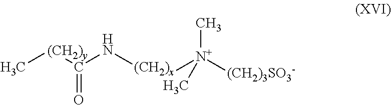

wherein x=1-4 and y=10-24.

In yet a further aspect the invention encompasses a method of treating obesity in a subject in need thereof comprising administering a pharmaceutically effective amount of a compound of formula (XVII) to the subject

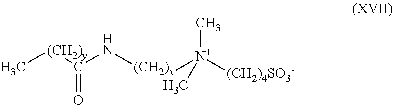

wherein x=1-4 and y=10-24.

In a further aspect the invention provides a method of treating obesity in a subject in need thereof comprising administering a pharmaceutically effective amount of a compound of formula (X) to the subject

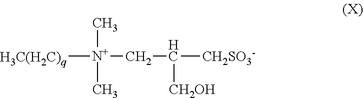

wherein q=14-20.

In another aspect of the invention there is provided a method of treating obesity in a subject in need thereof comprising administering a pharmaceutically effective amount of a compound of formula (III)

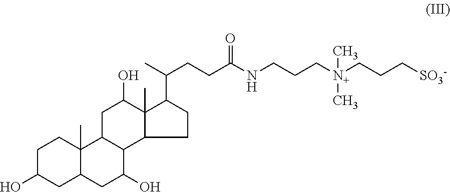

to the subject.

The invention further provides a method of treating obesity in a subject in need thereof comprising administering a pharmaceutically effective amount of a compound of formula (IX):

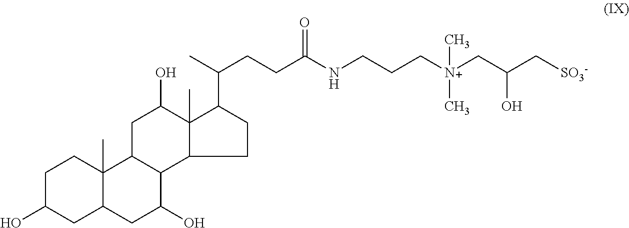

to the subject.

By another one of its aspects the invention envisages a method of treating age-related macular degeneration in a subject in need thereof comprising administering a pharmaceutically effective amount of a compound of formula (II) to the subject

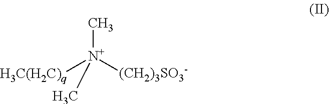

wherein q=14-20.

By another one of its aspects the invention envisages a method of treating age-related macular degeneration in a subject in need thereof comprising administering a pharmaceutically effective amount of a compound of formula (XI) to the subject

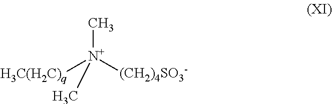

wherein q=14-20.

In a further aspect the invention encompasses a method of treating age-related macular degeneration in a subject in need thereof comprising administering a pharmaceutically effective amount of a compound of formula (XVI) to the subject

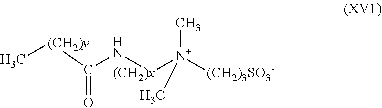

wherein x=1-4 and y=10-24.

In yet a further aspect the invention encompasses a method of treating age-related macular degeneration in a subject in need thereof comprising administering a pharmaceutically effective amount of a compound of formula (XVII) to the subject

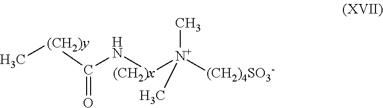

wherein x=1-4 and y=10-24.

The invention further provides a method of treating age-related macular degeneration in a subject in need thereof comprising administering a pharmaceutically effective amount of a compound of formula (X) to the subject

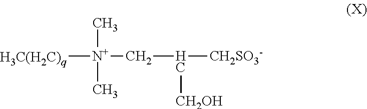

wherein q=14-20.

In another one of its aspects the invention provides a method of treating age-related macular degeneration in a subject in need thereof comprising administering a pharmaceutically effective amount of a compound of formula (III):

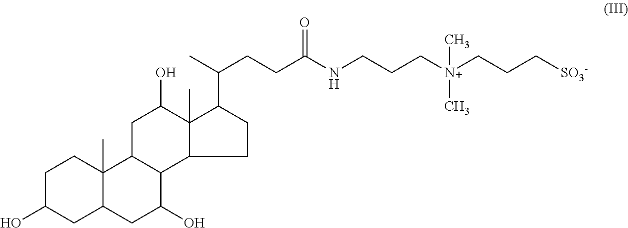

to the subject.

By another one of its aspects the invention envisages a method of treating age-related macular degeneration in a subject in need thereof comprising administering a pharmaceutically effective amount of a compound of formula (IX):

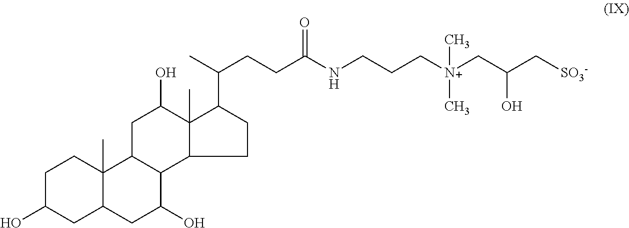

to the subject.

The invention provides a method of treating a neurodegenerative disease in a subject in need thereof comprising administering a pharmaceutically effective amount of a compound of formula (II) to the subject

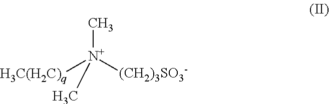

wherein q=14-20.

The invention further provides a method of treating a neurodegenerative disease in a subject in need thereof comprising administering a pharmaceutically effective amount of a compound of formula (XI) to the subject

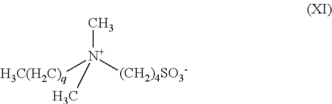

wherein q=14-20.

In a further aspect the invention encompasses a method of treating a neurodegenerative disease in a subject in need thereof comprising administering a pharmaceutically effective amount of a compound of formula (XVI) to the subject

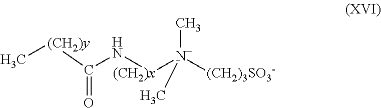

wherein x=1-4 and y=10-24.

In yet a further aspect the invention encompasses a method of treating a neurodegenerative disease in a subject in need thereof comprising administering a pharmaceutically effective amount of a compound of formula (XVII) to the subject

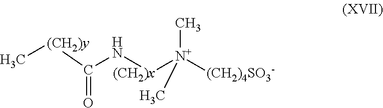

wherein x=1-4 and y=10-24.

In another aspect the invention envisages a method of treating a neurodegenerative disease in a subject in need thereof comprising administering a pharmaceutically effective amount of a compound of formula (X) to the subject

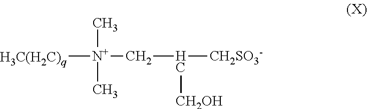

wherein q=14-20.

In a further aspect the invention encompasses a method of treating a neurodegenerative disease in a subject in need thereof comprising administering a pharmaceutically effective amount of a compound of formula (III):

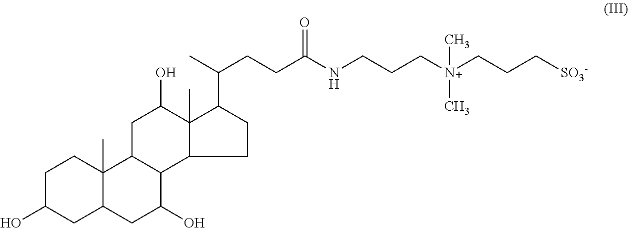

to the subject.

The invention further provides a method of a neurodegenerative disease in a subject in need thereof comprising administering a pharmaceutically effective amount of a compound of formula (IX):

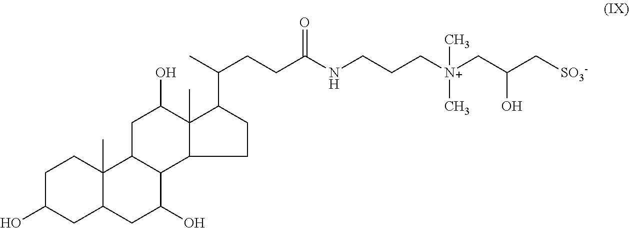

to the subject.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 demonstrates the effect of SB-18 alone and in conjunction with Hamsa treatment (cimetidine, sulfasalazine, diclofenac and cyclophosphamide) on tumor growth.

FIG. 2 demonstrates the effect of CHAPS alone and in conjunction with Hamsa treatment (cimetidine, sulfasalazine, diclofenac and cyclophosphamide) on tumor growth.

FIG. 3 demonstrates the effect of low dose SB-12 alone and in conjunction with Hamsa treatment (cimetidine, sulfasalazine, diclofenac and cyclophosphamide) on tumor growth.

FIG. 4 demonstrates the effect of high dose SB-12 in conjunction with Hamsa treatment (cimetidine, sulfasalazine, diclofenac and cyclophosphamide) on tumor growth.

FIG. 5 represents the ¹H-NMR spectrum of SB-16-4 in CDCl₃.

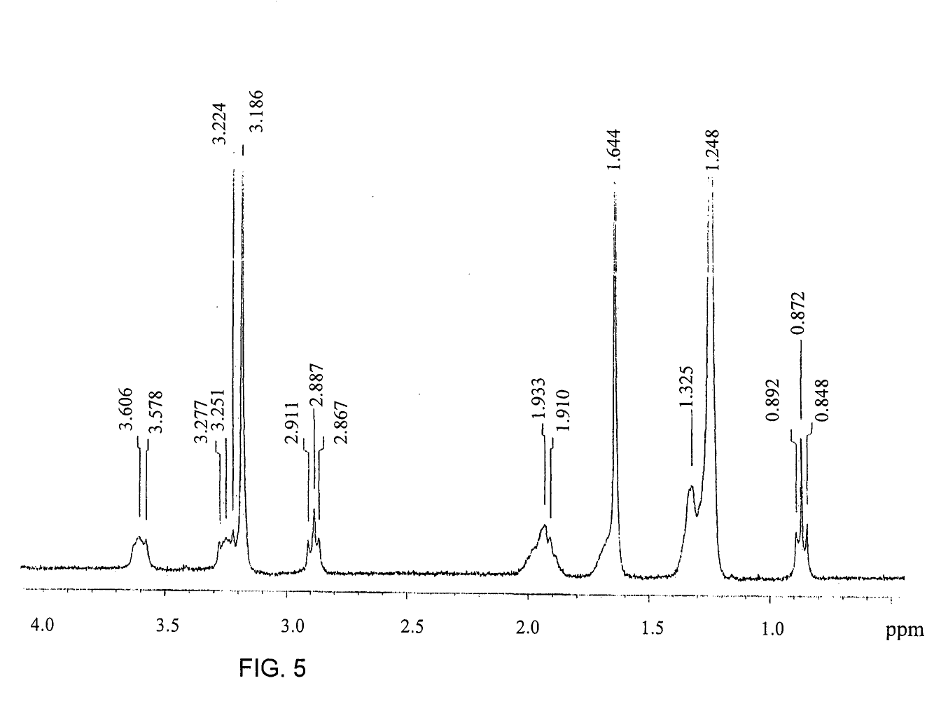

FIG. 6 demonstrates the effect of SB-16-4 alone and in conjunction with Hamsa treatment (cimetidine, sulfasalazine, diclofenac and cyclophosphamide) on tumor growth.

FIG. 7 demonstrates the effect of SB-18-4 alone and in conjunction with Hamsa treatment (cimetidine, sulfasalazine, diclofenac and cyclophosphamide) on tumor growth.

### DETAILED DESCRIPTION OF THE INVENTION

The subject invention provides a pharmaceutical composition comprising a sulfobetaine (SB) of general formula (I)

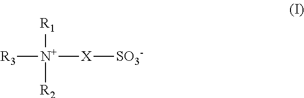

wherein
   X is —(CH₂)ₙ—, wherein n is 1-4, optionally substituted by at least one group selected from —(CH₂)ₚCH₃, —(CH₂)ₚOH, —(CH₂)ₚNH₂, and —(CH₂)ₚSH wherein p is 0-3;
   R₁ and R₂ independently of each other are each selected from a group consisting of H and —(CH₂)ₘCH₃ wherein m is 0-3; or
   R₁ and R₂ form together with the nitrogen atom a 5-8 membered hetero-aliphatic or hetero-aromatic ring;
   R₃ is a straight or branched C₁₄-C₂₅ alkyl, straight or branched C₁₄-C₂₅ alkenyl, straight or branched C₁₄-C₂₅ alkynyl, each optionally substituted with at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkylamino, dialkylamino, carboxyl, or thio; and each optionally interrupted by at least one group selected from —C(═O)—NH—, —C(═S)—NH—, —C(═O)—SH—, —C(═O)—O—, —NH—C(═O)NH— and —NH—C(═S)NH—;
   or R₃ is a group having a formula (VIII):

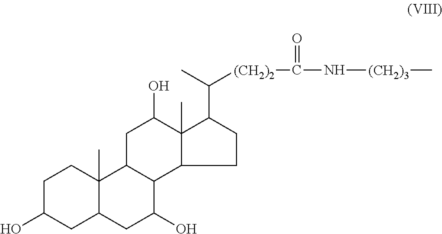

or enantiomers or diastereomers thereof; and a pharmaceutically acceptable carrier.

In one embodiment, when p equals zero (0), the at least one group optionally substituted on —(CH₂)ₙ—, is selected from —CH₃, —OH, —NH₂, and —SH. In another embodiment when m equals 0, R₁ and/or R₂ are —CH₃.

In one embodiment X is —(CH₂)ₙ— wherein n is 1-4.

In another embodiment, X is —(CH₂)ₙ— wherein n is 3, R₁ and R₂ are CH₃, and R₃ is —(CH₂)_{q}CH₃, wherein q=14-20. In a further embodiment q equals 15. In yet a further embodiment q equals 17.

In another embodiment, X is —(CH₂)ₙ— wherein n is 4, R₁ and R₂ are CH₃, and R₃ is —(CH₂)_{q}CH₃, wherein q=14-20. In a further embodiment q equals 15. In yet a further embodiment q equals 17.

In another embodiment, X is —(CH₂)ₙ— wherein n is 3, R₁ and R₂ are CH₃, and R₃ is —(CH₂)_{X}NH—C(═O)—(CH₂)_{y}CH₃, wherein x=1-4 and y=10-24. In a further embodiment x equals 3 and y equals 16.

In yet a further embodiment X is —(CH₂)ₙ—, wherein n is 4, R₁ and R₂ are CH₃, and R₃ is —(CH₂)ₓ—NH—C(═O)—(CH₂)_{y}CH₃, wherein x=1-4 and y=10-24. In another embodiment x equals 3 and y equals 16.

In another embodiment R₁ and R₂ are CH₃, and R₃ is —(CH₂)_{q}CH₃, wherein q=14-20 and X is

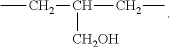

In one embodiment a composition of the invention comprises a sulfobetaine named CHAPS (3-[3-(Cholamidopropyl)dimethylamonio]-1-propanesulfonate) having the formula (III):

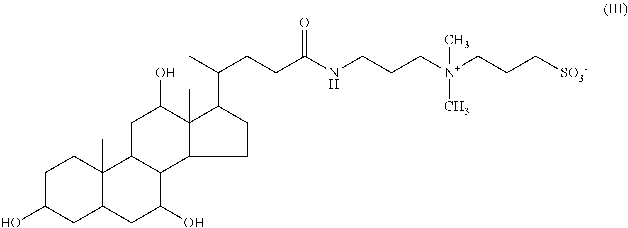

In another embodiment a composition of the invention comprises a sulfobetaine named CHAPSO (3-[3-(Cholamidopropyl)dimethylamonio]-2-hydroxypropane sulfonate) having the formula (IX):

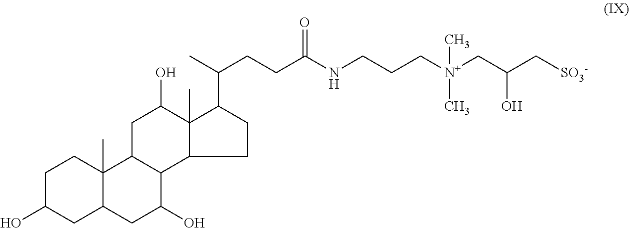

The term “SB” as used herein refers to any sulfobetaine. The SB number designates the number of carbons in a linear alkyl hydrophobic tail. Non-limiting examples of such sulfobetaines are SB-8, SB-10, SB-12, SB-14, SB-16, SB-18, SB-20, SB-16-4, SB-18-4, SB-20AM, SB-20-4AM and so forth. The synthesis of a variety of other SBs, such as, but not limited to, farnesyl-sulfabetaine, is described in Spencer T A. et al. J. Org. Chem. 64: 807-18 (1999). In addition, the term “SB” as used herein also refers to a molecule named CHAPS, also named 3-[3-(Cholamidopropyl)dimethylamonio]-1-propanesulfonate (Hjelmeland L M Proc. Natl. Acad. Sci. USA 77: 6368-70 (1980)) having a structural formula III and also to its 2-hydroxy-propanesulfonate derivative known as CHAPSO having structural formula IX.

The term “SB-12”, also named N-Dodecyl-N,N-Dimethyl-3-Ammonio-1-Propane Sulfonate, as used herein is a compound of formula (IV):

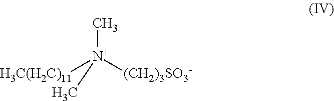

The term “SB-14”, also named N-Tetradecyl-N,N-Dimethyl-3-Ammonio-1-Propane-Sulfonate as used herein is a compound of formula (V):

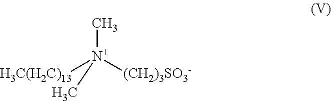

The term “SB-16”, also named N-Hexadecyl-N,N-Dimethyl-3-Ammonio-1-Propane-Sulfonate, as used herein is a compound of formula (VI):

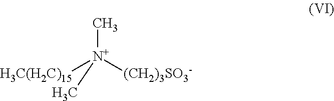

The term “SB-18”, also named N-Octadecyl-N,N-Dimethyl-3-Ammonio-1-Propane Sulfonate, as used herein is a compound of formula (VII):

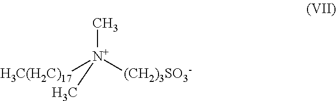

The term “SB-20”, also named N-Decadecyl-N,N-Dimethyl-3-Ammonio-1-Propane Sulfonate, as used herein is a compound of formula (VIII):

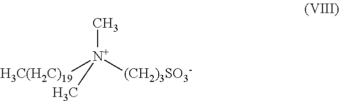

The term “SB-16-4”, also named N,N-Dimethylhexadecyl-ammonium-1-(4-butylsulfonate, as used herein is a compound of formula (XII):

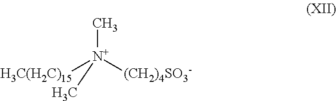

The term “SB-18-4”, also named N,N-Dimethyloctadecyl-ammonium-1-(4-butylsulfonate, as used herein is a compound of formula (XIII)

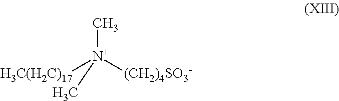

The term “SB-20AM”, also named 3-[N,N-Dimethyl-(3-octadecanoylaminopropyl)ammonio]-propanesulfonate, as used herein is a compound of formula (XIV)

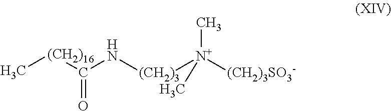

The term “SB-20-4AM”, also named 3-[N,N-Dimethyl-(3-octadecanoylaminopropyl)ammonio]-butanesulfonate, as used herein is a compound of formula (XV)

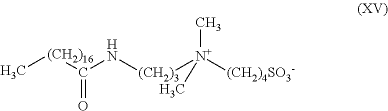

The term “cytotoxic agent” as used herein should be understood to encompass any agent used for the treatment of abnormal and uncontrolled progressive cellular growth. A cytotoxic agent acts as an angiogenesis inhibitor when administered at a low dose. Non limiting examples of cytotoxic agents include the alkylating agents cyclophosphamide (CTX) (Bristol-Meyers Squibb), ifosfamide (Bristol-Meyers Squibb), chlorambucil (Glaxo Wellcome), and carmustine (Bristol-Meyers Squibb); the anti-metabolites cytarabine (Pharmacia & Upjohn), 6-mercaptopurine (Glaxo Wellcome), 6-thioguanine (Glaxo Wellcome), and methotrexate (Immunex); the antibiotics doxorubicin (Pharmacia & Upjohn), daunorubicin (NeXstar), and mitoxantrone (Immunex); and miscellaneous agents such as vincristine (Lilly), vinblastine (Lilly), and paclitaxel (Bristol-Meyers Squibb) or their pharmaceutically acceptable salts.

In one embodiment a pharmaceutical composition of the invention further comprises a cytotoxic agent. In one embodiment a cytotoxic agent is selected from the group consisting of: cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, and paclitaxel or their pharmaceutically acceptable salts. In a further embodiment, the cytotoxic agent is cyclophosphamide.

The term “anti-inflammatory agent (drug)” as used herein should be understood to encompass any agent capable of reducing and/or inhibiting and/or preventing inflammation disease caused for instance by a response to infection, injury, irritation, or surgery. The anti-inflammatory agent may be a steroidal or a non-steroidal anti-inflammatory agent. Non-limiting examples of steroidal anti-inflammatory drugs are dexamethasone and betamethasone. Non-limiting examples of a non-steroidal anti-inflammatory drugs are a COX-1 inhibitor, a COX-2 inhibitor and a non-selective COX-1 and COX-2 inhibitor. Non-limiting examples of COX-1 and COX-2 inhibitors are diclofenac, piroxicam and indomethacin.

In one embodiment a pharmaceutical composition of the invention further comprises an anti-inflammatory agent. In one embodiment an anti-inflammatory agent is selected from the group consisting of steroidal and non-steroidal anti-inflammatory agents.

In a further embodiment, an anti-inflammatory agent is a non-steroidal anti-inflammatory agent. In yet a further embodiment the non-steroidal anti-inflammatory agent is selected from the group consisting of COX-1 and COX-2 inhibitors. In another embodiment the COX1/COX2 inhibitor is selected from the group consisting of diclofenac, piroxicam and indomethacin. In yet a further embodiment of the invention, the anti-inflammatory agent is diclofenac.

In a further embodiment of the invention an anti-inflammatory agent is a steroidal anti-inflammatory agent. In one embodiment the steroidal anti-inflammatory agent is dexamethasone or betamethasone.

The term “NFkB inhibitor” as used herein should be understood to encompass any agent used for the inhibition of the Nuclear Factor kappa B (NFkB) intracellular transcription factor. Non-limiting examples of an NFkB inhibitor are sulfasalazine, rapamycin, caffeic acid phenethylester, SN50 (a cell-permeable inhibitory peptide), parthenolide, triptolide, wedelolactone, lactacystin and MG-132 [Z-Leu-Leu-Leu-H] or derivatives thereof. For example, temsirolimus and everolimus (derivatives of rapamycin).

In one embodiment a pharmaceutical composition of the invention further comprises an NFκB inhibitor. In one embodiment the NFkB inhibitor is sulfasalazine or rapamycin. In yet a further embodiment the NFkB inhibitor is sulfasalazine.

The term “H2-blocker” as used herein should be understood to encompass any histamine type 2-receptor antagonist used to block the action of histamine on parietal cells in the stomach and decreasing acid production by these cells. Non-limiting examples of such H2-blocker are cimetidine, ranitidine, famotidine and nizatidine.

In one embodiment a pharmaceutical composition of the invention further comprises an H2-blocker. In one embodiment the H2-blocker is selected from the group consisting of cimetidine, ranitidine, famotidine and nizatidine. In yet a further embodiment the H2 blocker is cimetidine.

The invention further provides a sulfobetaine compound of formula (I)

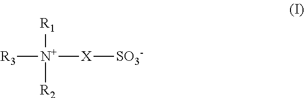

wherein X, R₁, R₂ and R₃ are as defined above, or enantiomers or diastereomers thereof and a pharmaceutically acceptable carrier for therapy.

In a further aspect there is provided a use of a sulfobetaine of formula (I)

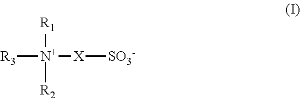

wherein X, R₁, R₂ and R₃ are as defined above, or enantiomers or diastereomers thereof and a pharmaceutically acceptable carrier for the manufacture of a medicament.

In one embodiment the medicament is for the treatment of cancer.

The term “cancer” as used herein should be understood to encompass any neoplastic disease which is characterized by abnormal and uncontrolled cell division causing malignant growth or tumor. Cancer cells, unlike benign tumor cells, exhibit the properties of invasion and metastasis and are highly anaplastic. Cancer includes the two broad categories of carcinoma and sarcoma. Cancer as used herein may refer to either a solid tumor or tumor metastasis. Non-limiting examples of cancer are lung cancers (e.g. adenocarcinoma and including non-small cell lung cancer), pancreatic cancers (e.g. pancreatic carcinoma such as, for example exocrine pancreatic carcinoma), colon cancers (e.g. colorectal carcinomas, such as, for example, colon adenocarcinoma and colon adenoma), prostate cancer including the advanced disease, hematopoietic tumors of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumors of mesenchymal origin (e.g. fibrosarcomas and rhabdomyosarcomas), melanomas, teratocarcinomas, neuroblastomas, gliomas, glioblastoma, benign tumor of the skin (e.g. keratoacanthomas), breast carcinoma (e.g. advanced breast cancer), kidney carcinoma, ovary carcinoma, bladder carcinoma, epidermal carcinoma, cervical cancer, endometrial cancer, anaplastic large cell lymphoma, esophageal squamous cells carcinoma, hepatocellular carcinoma, follicular dendritic cell carcinoma, intestinal cancer, muscle-invasive cancer, seminal vesicle tumor, epidermal carcinoma and so forth.

The term “inhibiting cancer” or “treating cancer” as used herein should be understood to encompass a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer. The term “inhibiting cancer” and “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer.

The term “anti-cancer agent” as used herein should be understood to encompass any drug or treatment effective to treat or inhibit cancer. Non-limiting examples of an anti-cancer agent are cytotoxic drugs, cytostatic drugs, anti-angiogenic drugs or drugs that deprive the cancer cells of an essential growth factor or hormone.

In a further embodiment the medicament is for the treatment of obesity.

The term “obesity” as used herein should be understood to encompass a condition in which the natural energy reserve, stored in the fatty tissue of a mammal, exceeds healthy limits. Mammals suffering from an obese condition may be predisposed to various diseases, such as for example cardiovascular diseases, diabetes mellitus type 2, sleep apnea and osteoarthritis. Obese conditions are individually defined by different classifications, for example obese condition may be defined as a body mass index (weight divided by height squared) of 30 kg/m2 or higher. Another definition may relate to “central obesity” defined by the waist circumference (>102 cm in men and >88 cm in women) or waist-hip ratio (>0.9 for men and >0.85 for women). In yet a further definition, obesity may be defined by percent of body fat (men with more than 25% body fat and women with more than 30% body fat).

In yet another embodiment the medicament is for the treatment of age-related macular degeneration.

The term “age-related macular degeneration (AMD)” should be understood to encompass a medical condition in which the center of the inner lining of the eye, known as the macula area of the retina, suffers thickening, atrophy, and in some cases, watering. This condition can result in loss of side vision, which entails inability to see coarse details, to read, or to recognize faces.

In another embodiment the medicament is for the treatment of a neurodegenerative disease.

The term “neurodegenerative disease” should be understood to encompass any disease or condition in which cells of the brain and spinal cord are lost or damaged. Neurodegenerative diseases result from deterioration of neurons or their myelin sheath which over time will lead to dysfunction and disabilities, for example problems with movements (i.e. ataxia) and/or problems with cognitive function such as memory, attention and learning (i.e. dementia).

Non-limiting examples of neurodegenerative diseases are Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis and so forth.

“A subject” as used herein can be a male or a female subject; A subject can be a human being or any other mammal.

Suitable routes of administration of a pharmaceutical composition of the subject invention are oral, rectal, nasal, topical (including transdermal, buccal and sublingual), vaginal or parenteral (including subcutaneous, intramuscular, intravenous and intradermal) administration or administration via an implant. In a specific embodiment, a pharmaceutical composition of the invention is administered orally.

The exact dose and regimen of administration of a pharmaceutical composition of the invention will necessarily be dependent upon the therapeutic effect to be achieved (e.g. treatment of cancer) and may vary with the particular compound(s), the route of administration, and the age and condition of the individual subject to whom the medicament is to be administered.

A dosage for humans is likely to contain 0.1-100 mg per kg body weight per day. The desired dose may be presented as one dose or as multiple sub-doses administered at appropriate intervals.

In the context of the present invention the term “pharmaceutically acceptable carrier” relates to pharmaceutically-acceptable, nontoxic carriers or diluents, which are defined as vehicles commonly used to formulate pharmaceutical compositions for animal or human administration. Such carriers may include, however not limited to, buffering agents, solubilizing agents, stabilizing agents or taste additives.

The present invention thus relates to a pharmaceutical composition of the subject invention in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association a compound of the invention with any auxiliary agent. The auxiliary agent(s), also named accessory ingredient(s), include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units (dosage forms) such as pills, tablets, dragées or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The compositions of the invention may be administered in conjunction with other compounds, including, but not limited to, estrogens, androgens, progestagens, antiprogestagens, immunological modifiers such as interferons and interleukins, growth hormones or other cytokines, folic acid, vitamins, minerals and so forth, and/or in combination with surgery and/or radiation therapy.

A pharmaceutical composition of the invention may be administered in a single dosage form comprising all active ingredients; or may be administered in separate dosage forms each containing one or more active ingredients. When a pharmaceutical composition of the invention is administered in more than one dosage form, these dosage forms can be administered by the same or by distinct routes of administration. When a pharmaceutical composition of the invention is administered in more than one dosage form, these dosage forms can be administered simultaneously or sequentially.

The invention further provides a method of treating cancer comprising administering a pharmaceutical composition of the invention.

The invention further provides a method of treating obesity comprising administering a pharmaceutical composition of the invention.

In yet a further aspect the invention provides a method of treating age related macular degeneration comprising administering a pharmaceutical composition of the invention.

In a further aspect the invention provides a method of treating a neurodegenerative disease comprising administering a pharmaceutical composition of the invention.

In one aspect the invention provides a method of treating cancer in a subject in need thereof comprising administering a pharmaceutically effective amount of a compound of formula (II) to the subject

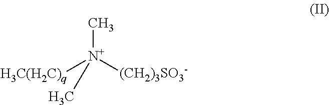

wherein q=14-20.

In one embodiment, q equals 15. In a further embodiment q equals 17.

In another aspect the invention provides a method of treating cancer in a subject in need thereof comprising administering a pharmaceutically effective amount of a compound of formula (XI) to the subject

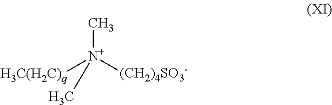

wherein q=14-20.

In one embodiment, q equals 15. In a further embodiment q equals 17.

In a further aspect the invention encompasses a method of treating cancer in a subject in need thereof comprising administering a pharmaceutically effective amount of a compound of formula (XVI) to the subject

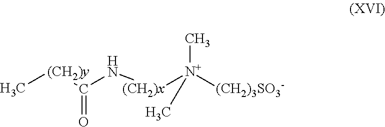

wherein x=1-4 and y=10-24.

In one embodiment x equals 3 and y equals 16.

In yet a further aspect the invention encompasses a method of treating cancer in a subject in need thereof comprising administering a pharmaceutically effective amount of a compound of formula (XVII) to the subject

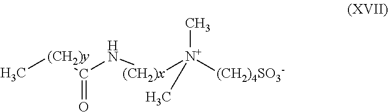

wherein x=1-4 and y=10-24.

In one embodiment x equals 3 and y equals 16.

In another aspect the invention provides a method of treating cancer in a subject in need thereof comprising administering a pharmaceutically effective amount of a compound of formula (X) to the subject

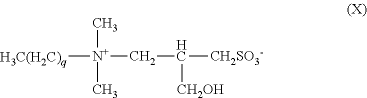

wherein q=14-20.

In a further aspect of the invention there is provided a method of treating cancer in a subject in need thereof comprising administering a pharmaceutically effective amount of a compound of formula (III):

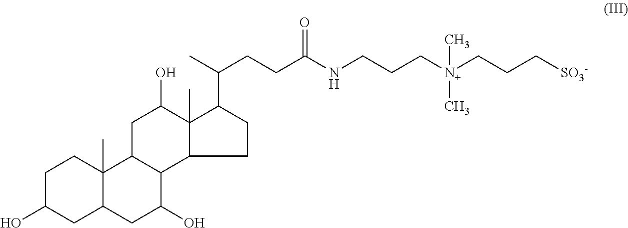

to the subject.

In another aspect of the invention there is provided a method of treating cancer in a subject in need thereof comprising administering a pharmaceutically effective amount of a compound of formula (IX):

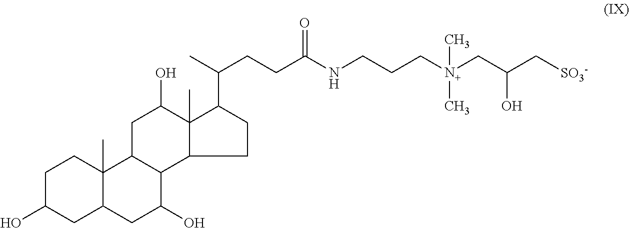

to the subject.

In yet a further embodiment, an additional anti-cancer agent may be administered in conjunction with a composition of the invention.

In another aspect the invention provides a method of treating obesity in a subject in need thereof comprising administering a pharmaceutically effective amount of a compound of formula (II) to the subject

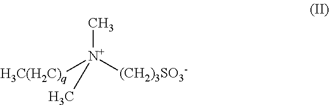

wherein q=14-20.

In one embodiment q equals 15. In a further embodiment q equals 17.

In another aspect the invention provides a method of treating obesity in a subject in need thereof comprising administering a pharmaceutically effective amount of a compound of formula (XI) to the subject

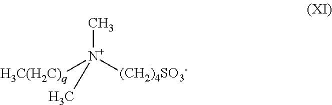

wherein q=14-20.

In one embodiment, q equals 15. In a further embodiment q equals 17.

In a further aspect the invention encompasses a method of treating obesity in a subject in need thereof comprising administering a pharmaceutically effective amount of a compound of formula (XVI) to the subject

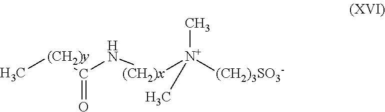

wherein x=1-4 and y=10-24.

In one embodiment x equals 3 and y equals 16.

In yet a further aspect the invention encompasses a method of treating obesity in a subject in need thereof comprising administering a pharmaceutically effective amount of a compound of formula (XVII) to the subject

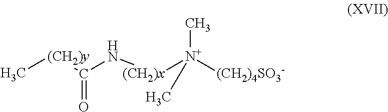

wherein x=1-4 and y=10-24.

In one embodiment x equals 3 and y equals 16.

The invention further provides a method of treating obesity in a subject in need thereof comprising administering a pharmaceutically effective amount of a compound of formula (X) to the subject

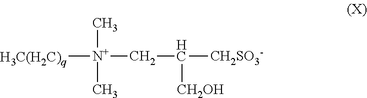

wherein q=14-20.

In another one of its aspects the invention provides a method of treating obesity in a subject in need thereof comprising administering a pharmaceutically effective amount of a compound of formula (III):

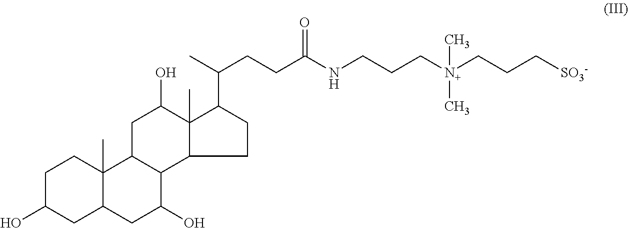

to the subject.

In a further aspect the invention envisages a method of treating obesity in a subject in need thereof comprising administering a pharmaceutically effective amount of a compound of formula (IX):

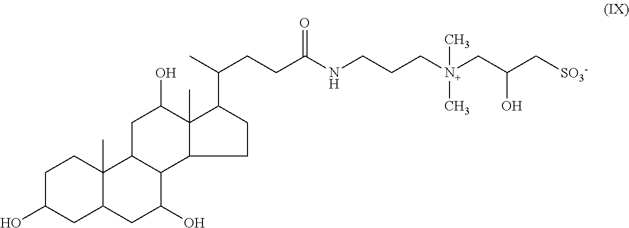

to the subject.

In yet one further aspect the invention provides a method of treating age-related macular degeneration in a subject in need thereof comprising administering a pharmaceutically effective amount of a compound of formula (II) to the subject

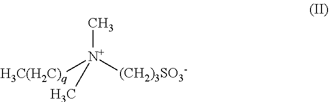

wherein q=14-20.

In one embodiment q equals 15. In a further embodiment q equals 17.

In another aspect the invention provides a method of treating age-related macular degeneration in a subject in need thereof comprising administering a pharmaceutically effective amount of a compound of formula (XI) to the subject

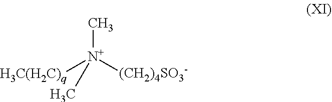

wherein q=14-20.

In one embodiment, q equals 15. In a further embodiment q equals 17.

In a further aspect the invention encompasses a method of treating age-related macular degeneration in a subject in need thereof comprising administering a pharmaceutically effective amount of a compound of formula (XVI) to the subject

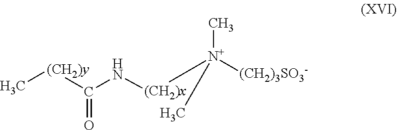

wherein x=1-4 and y=10-24.

In one embodiment x equals 3 and y equals 16.

In yet a further aspect the invention encompasses a method of treating age-related macular degeneration in a subject in need thereof comprising administering a pharmaceutically effective amount of a compound of formula (XVII) to the subject

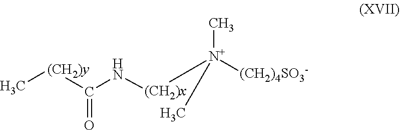

wherein x=1-4 and y=10-24.

In one embodiment x equals 3 and y equals 16.

In another aspect the invention provides a method of treating age-related macular degeneration in a subject in need thereof comprising administering a pharmaceutically effective amount of a compound of formula (X) to the subject

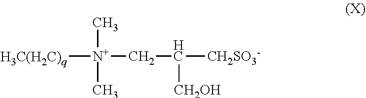

wherein q=14-20.

In one further aspect the invention provides a method of treating age-related macular degeneration in a subject in need thereof comprising administering a pharmaceutically effective amount of a compound of formula (III):

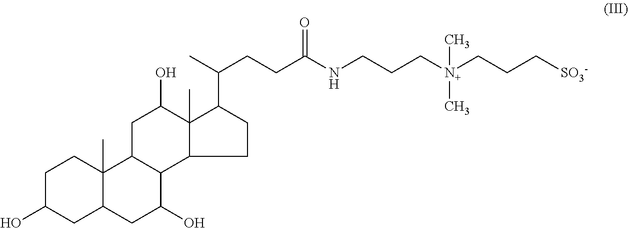

to the subject.

In another one of its aspects the invention provides a method of treating age-related macular degeneration in a subject in need thereof comprising administering a pharmaceutically effective amount of a compound of formula (IX):

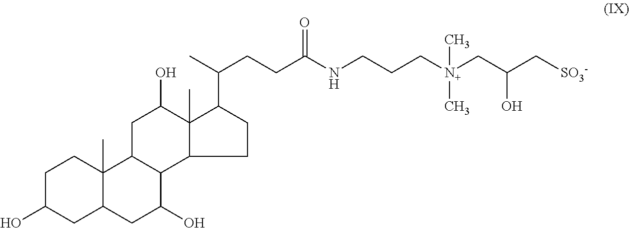

to the subject.

In yet a further aspect the invention envisages a method of treating a neurodegenerative disease in a subject in need thereof comprising administering a pharmaceutically effective amount of a compound of formula (II) to the subject

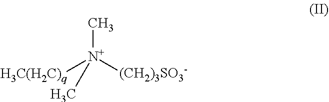

wherein q=14-20.

In one embodiment q equals 15. In a further embodiment q equals 17.

In another aspect the invention provides a method of treating a neurodegenerative disease in a subject in need thereof comprising administering a pharmaceutically effective amount of a compound of formula (XI) to the subject

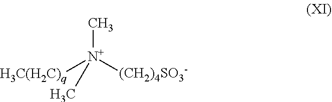

wherein q=14-20.

In one embodiment, q equals 15. In a further embodiment q equals 17.

In a further aspect the invention encompasses a method of treating a neurodegenerative disease in a subject in need thereof comprising administering a pharmaceutically effective amount of a compound of formula (XVI) to the subject

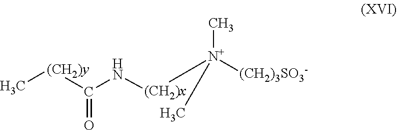

wherein x=1-4 and y=10-24.

In one embodiment x equals 3 and y equals 16.

In yet a further aspect the invention encompasses a method of treating a neurodegenerative disease in a subject in need thereof comprising administering a pharmaceutically effective amount of a compound of formula (XVII) to the subject

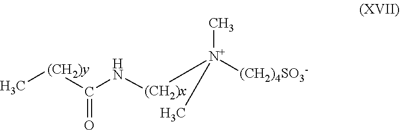

wherein x=1-4 and y=10-24.

In one embodiment x equals 3 and y equals 16.

In another one of its aspects the invention provides a method of treating a neurodegenerative disease in a subject in need thereof comprising administering a pharmaceutically effective amount of a compound of formula (X) to the subject

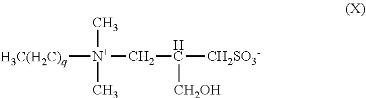

wherein q=14-20.

In one other aspect the invention encompasses a method of treating a neurodegenerative disease in a subject in need thereof comprising administering a pharmaceutically effective amount of a compound of formula (III)

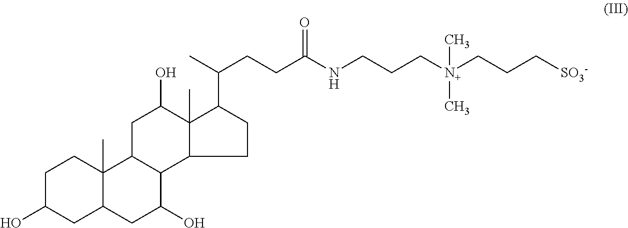

to the subject.

In a further aspect the invention provides a method of a neurodegenerative disease in a subject in need thereof comprising administering a pharmaceutically effective amount of a compound of formula (IX):

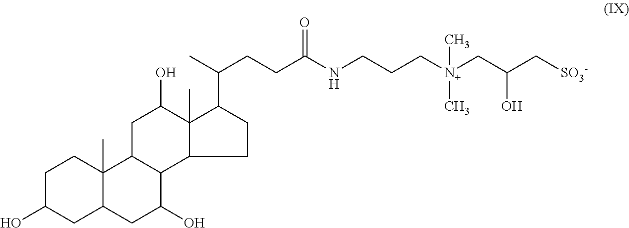

to the subject.

The term “alkyl” as used herein refers to a saturated aliphatic hydrocarbon having between about 1 and about 25 carbon atoms, which may be arranged as a straight (linear) chain or branched chain, or as a cyclic group. The term “C₁₄-C₂₅ alkyl” or “an alkyl of 14 to 25 carbon atoms” refers to carbon chains having between 14 and 25 carbon atoms.

The term “alkenyl” refers to a non-saturated aliphatic hydrocarbon having between about 1 and about 25 carbon atoms having at least one carbon-carbon double bond, which may be arranged as a straight chain or branched chain, or as a cyclic group. The term “C₁₄-C₂₅ alkenyl” or “an alkenyl of 14 to 25 carbon atoms” refers to carbon chains having between 14 and 25 carbon atoms.

The term “alkynyl” refers to a non-saturated aliphatic hydrocarbon having between about 1 and about 25 carbon atoms, having at least one carbon-carbon triple bond, which may be arranged as a straight chain or branched chain, or as a cyclic group. The term “C₁₄-C₂₅ alkynyl” or “an alkynyl of 14 to 25 carbon atoms” refers to carbon chains having between 14 and 25 carbon atoms.

A branched or straight aliphatic substituent may be unsubstituted or substituted with one or more of a variety of groups selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkyl or dialkylamino, carboxyl, thio, and so forth. When substituted by a terminal group, the C₁₄-C₂₅ alkyl is an alkylene having between about 14 and about 25 carbon atoms, the C₁₄-C₂₅ alkenyl is an alkenylene having between about 14 and about 25 carbon atoms and the C₁₄-C₂₅ alkynyl is an alkynylene having between about 14 and about 25 carbon atoms.

The term “aryl” as used herein refers to a group or part of a group having an aromatic system which may include a single ring or multiple aromatic rings fused or linked together where at least one part of the fused or linked rings forms the conjugated aromatic system e.g. having 6 to 14 carbon atoms. The aryl groups may for example include, but are not limited to, phenyl, naphthyl, biphenyl, anthryl, tetrahydronaphthyl, phenanthryl, indene, benzonaphthyl, fluorenyl, and carbazolyl. The aryl group may be substituted by one or more substituents such as halogen, hydroxyl, alkyloxy, alkylthio, arylthio, alkoxy, alkylcarbonyl, carbonyl, alkoxycarbonyl, ester, amido, alkylamido, dialkylamido, benzyl, aryloxy, nitro, amino, alkyl or dialkylamino, carboxyl, thio, and so forth.

The term “alkoxy” as used herein refers to the —O-(alkyl) group, where the point of attachment is through the oxygen-atom and the alkyl group is as defined hereinbefore.

The term “aryloxy” as used herein refers to the —O-(aryl) group, where the point of attachment is through the oxygen-atom and the aryl group is as defined hereinbefore.

The term “alkyloxy” includes hydroxyalkyl and as used herein refers to the -alkyl-OH group, where the point of attachment is through the alkyl group which is defined as hereinbefore.

The term “alkylthio” refers similarly to -alkyl-SH.

The term “arylthio” as used herein refers to the —S-(aryl) group, where the point of attachment is through the sulfur-atom and the aryl group is as defined hereinbefore.

The term “alkylcarbonyl” as used herein refers to the —C═(O)-(alkyl) group, where the point of attachment is through the carbon-atom of the carbonyl moiety and the alkyl group is as defined hereinbefore.

The term “amino” refers to primary, secondary and tertiary amines where the point of attachment is through the nitrogen-atom. In case of the secondary or tertiary amines, the substituting groups on the nitrogen may be the same or different. In some embodiments, the amine group is a quaternary amine carrying a positive charge. In such embodiments, the charged amine is neutralized by a counter ion, as may be known to a person skilled in the art. When one group on the amino is H and the other is R (wherein R may be an alkyl), the group refers to “alkylamino”. When two R groups (which may be the same or different) are substituted on the nitrogen atom, the group refers to a “dialkylamino”.

The term “halogen” or “halo” as used herein refers to —Cl, —Br, —F, or —I atoms.

The term “ester” as used herein refers to a —C═(O)—OR, where the point of attachment are through both the C-atom of the ester group and R may be an alkyl group as defined herein above. One or both oxygen atoms of the ester group can be replaced with a sulfur atom, thereby forming a “thioester”, i.e., a —C═(O)—SR, —C═(S)—OR or —C═(S)—SR group.

The term “carbonyl” refers to the group —C═(O) wherein the point of attachment is through the carbon atom. Such carbonyls may form ketones, i.e. a RC═(O) group where the R group may be an alkyl as defined hereinabove, aldehyde, i.e. wherein in the group R is H.

The term “benzyl” refers to the group —CH₂C₆H₅.

The term “carboxyl” refers to the group —C═(O)—OH, wherein the point of attachment is through the carboxyl carbon atom.

The term “hydroxyl” refers to —OH, and “thio” refers to —SH.

The term “amido” refers to the group —C═(O)—NRR, wherein the points of attachments are through the carbon atom on one hand, and through the nitrogen atom on the other, wherein both R groups are H. When one group is H and the other is an alkyl, i.e., —C═(O)—NHR, the group refers to “alkylamido”. When both Rs are alkyls, the group refers to a “dialkylamido”.

The term “nitro” refers to the group —NO₂.

As used herein, the term “hetero-aromatic ring” should be understood to encompass a monocyclic or multicyclic aromatic ring system having between 5 to 8 atoms, which can be formed by R₁ and R₂ together with the nitrogen atom of sulfobetaine (SB) of general formula (I), wherein one or more additional atoms in the ring system can be a heteroatom, that is, an element other than carbon, including but not limited to, nitrogen, oxygen or sulfur.

As used herein, the term “hetero-aliphatic ring” should be understood to encompass a monocyclic or multicyclic non-aromatic ring system (which may be saturated, i.e. having only single bonds, or unsaturated, i.e. comprising at least one double and/or triple bond), having between 5 to 8 atoms, which can be formed by R₁ and R₂ together with the nitrogen atom of sulfobetaine (SB) of general formula (I), wherein one or more additional atoms in the ring system may be a heteroatom, that is, an element other than carbon, including but not limited to, nitrogen, oxygen or sulfur.

Non limiting examples of heteroaliphatic rings are pyrrolidinium ring, piperidinium ring, azepanium ring, morpholinium ring, oxazinium ring, oxazolidinium ring, thiomorpholinium ring, thiazinium ring, thiazolidinium ring, azepanium ring, oxazepanium ring, diazepanium ring and thiazepanium ring.

Non limiting examples of heteroaromatic rings are pyrrolium ring, pyridinium ring, azepinium ring, oxazepinium ring, diazepinium ring and thiazepinium ring.

The term “interrupted by at least one group selected from —C(═O)—NH—, —C(═S)—NH—, —C(═O)—SH—, —C(═O)—O—, —NH—C(═O)NH— and —NH—C(═S)NH—” is meant to encompass a straight or branched C₁₄-C₂₅ alkyl, straight or branched C₁₄-C₂₅ alkenyl, straight or branched C₁₄-C₂₅ alkynyl having at least one bivalent functionality selected from a group consisting of —C(═O)—NH—, —C(═S)—NH—, —C(═O)—SH—, —C(═O)—O—, —NH—C(═O)NH— and —NH—C(═S)NH— substituted between any two carbons within the straight or branched alkyl, alkenyl or alkynyl chain.

It should be understood that when R₁ is different than R₂, a sulfobetaine of the present invention may contain at least one chiral center at the quarternary nitrogen atom. Compounds of the invention may contain one or more additional chiral centers, and thus may exist in, and be isolated as, enantiomeric or diastereomeric forms, as racemic or non-racemic mixtures. The present invention includes any possible single enantiomers, diastereomers, racemates or mixtures of enantiomers or diastereomers, of any compound of the general Formula (I).

The invention is further described in the following examples, which are not in any way intended to limit the scope of the invention as claimed.

### EXAMPLES

### Example 1

### Synthesis of (SB-16-4) N,N-Dimethylhexadecyl-ammonium-1-(4-butylsulfonate)

2.70 gr (10 mmole) of N,N-Dimethylhexadecyl amine were dissolved in 100 ml dichloromethane, in a 250 ml round bottom flask. 1.63 gr (12 mmole) of 1,4-butansultone were added slowly to the magnetically stirred solution. The mixture was stirred overnight and the solvent was evaporated in a rotavapor. The residue was crystallized from 75 ml acetonitrile. 2.85 gr. (70% yield) were obtained.

### Example 2

### Synthesis of (SB-18-4) N,N-Dimethyloctadecyl-ammonium-1-(4-butylsulfonate)

This compound was synthesized from N,N-Dimethyloctadecyl amine and 1,4-butansultone using the same procedure as for SB-16-4, 65% yield was obtained.

### Example 3

### Synthesis of (SB-20AM) 3-[N,N-Dimethyl-(3-octadecanoylaminopropyl)ammonio]-propanesulfonate

2.1 gr. (20 mmole) of 3-(Dimethylamino)-1-propylamine were dissolved in 100 ml dichloromethane, in a 250 ml round bottom flask. 6.1 gr. (20 mmole) of Stearoyl chloride were added dropwise through a dropping funnel into the magnetically stirred solution. The mixture was stirred overnight and the solution was transferred to a separatory funnel. 100 ml dichloromethane and 200 ml 1:1 solution of dichloromethahe:methanol were added and the solution was washed three times with 100 ml water. Dried over MgSO4, filtered and evaporated to dryness. 90% yield was obtained.

1.9 gr. (5 mmole) of the resulted N-[3-(dimethylamino)propyl]-octadecylamide were dissolved in 50 ml dichloroethane and transferred to a round bottom flask. 600 mg NaOH were added followed by 0.73 gr. (6 mmole) 1,3-propanesultone. The mixture was stirred under reflux during 48 h and then evaporated to dryness, redissolved in 100 ml 2:1 dichloromethane:methanol and washed 3 times with 30 ml 0.2N NaOH. The organic phase was dried over MgSO4, filtered and evaporated to dryness. 55% yield was obtained.

### Example 4

### Synthesis of (SB-20-4AM) 3-[N,N-Dimethyl-(3-octadecanoylaminopropyl) ammonio]-butanesulfonate

This compound was synthesized from 3-(Dimethylamino)-1-propylamine, Stearoyl chloride and 1,4-butansultone using the same procedure as for SB-20AM, 50% yield was obtained.

### Example 5

### Effect of SB-18, CHAPS, SB-12, SB-16-4, SB-18-4 (Alone or in Combination) on Tumor Growth of Malignant Tumors

In order to determine the effect of SB-18, CHAPS, SB-12, SB-16-4 and SB-18-4 each alone and each in combination with 4 other agents (“Hamsa treatment”), on tumor growth of a malignant (mammary carcinoma) tumor (EMT6/CTX) in a mouse experimental model, six (6) experiments were carried out as described below:

CB6F1 male mice (a cross between Balbc and C57bl) at age of 7-8 weeks were anesthetized with Ketamine:Xelazine in a 0.85:0.15 ratio respectively, and diluted 1:1 with double distilled water (DDW) and injected i.m. in volume of 50 uL.

100 uL of EMT6/CTX cells 3.6×10⁵ cells/mouse were injected subcutaneously (s.c.) to the anesthetized mice, after shaving their back (to enable accurate measurement of tumor-volume). The mice were divided into four groups, as outlined below, 6-8 animals in each group. The mice were marked with Picric acid on head (H), tail (T), head and tail (H&T), right leg (RL), left leg (LL), right and left legs (SY), back and blank and were individually monitored.

“Hamsa treatment” as used herein consists of the following active ingredients in the following concentrations:
Cimetidine 60 mg/kg (Farchemia s.r.l. Italy).
Diclofenac 30 mg/kg (Unique Pharma India).
Cyclophosphamide (CTX) 60 mg/kg (Hisun China)
Sulfasalazine (SSZ) (Farchemia s.r.l. Italy)—50 mg/kg on days of cytotoxic treatment, i.e. on days when cyclophosphamide and diclofenac are administered; 150 mg/kg on days of non-cytotoxic treatment, i.e. on days when no cyclophosphamide and disclofenac are administered.

The preparation and administration protocol of the Hamsa treatment is outlined in Table 1:

TABLE 1

| | | |
|---|---|---|
| Substance | Amount | pH |

| | | | |
|---|---|---|---|
| Preparation for non-cytotoxic days | | | |
| Vehicle (2% Solutol- | 80 | mL | |
| HS15 in DDW) | | | |
| Acetic Acid 10% | ~2 | mL | <3.0 |
| Cimetidine (12 mg/mL) | 0.96 | gram | |
| Na₂CO₃(0.066M) | 0.56 | gram | ~9 |
| SSZ (30 mg/mL) | 2.4 | gram | |
| Final pH | | | 6-8 |
| Preparation for cytotoxic days | | | |
| Vehicle (2% Solutol | 40 | mL | |
| in DDW) | | | |
| Acetic acid 10% | ~2 | mL | <3.0 |
| Cimetidine (12 mg/mL) | 0.48 | gram | |
| Na₂CO₃ (0.0044M) | 0.186 | gram | ~9 |
| SSZ (10 mg/mL) | 0.4 | gram | |
| Diclofenac (6 mg/mL) | 0.24 | gram | |
| Cyclophosphamide (12 mg/mL) | 0.48 | gram | |
| Final pH | | | 6-8 |

For compositions containing Cimetidine and Sulfasalazine: in order to increase Cimetidine solubility, the pH of the solution was brought to acidic ranges (pH-2). This was achieved by adding Acetic acid 10% to DDW adding about 3% to the solution volume. In order to increase Sulfasalazine solubility, the pH of the solution was brought to basic ranges (pH˜10.5). This was achieved by adding Na₂CO₃ to DDW to a concentration of 0.1M. Sulfasalazine was added and the pH neutralized. All other components were then added to the solution and stirred until solution was homogenous.

SB-12, CHAPS and SB-18 (HOPAX Chem. Mfg. Ltd Taiwan) were prepared separately as aqueous solutions, then mixed 1:1 with Vehicle or the cytotoxic or non-cytotoxic stage of the Hamsa treatment and twice the volume was injected i.p. Due to its lower solubility, SB-18 was dissolved by mild warming (˜45° C.) of its aqueous solution in the presence of 2% Solutol HS-15 (BASF-Germany). In groups that received SB-12, CHAPS or SB-18, this agent was injected daily.

SB-16-4 and SB-18-4 were prepared as described in Examples 1 and 2.

SB-16-4 was first dissolved in DDW containing 2% Solutol HS-15 with heating to 40° C. to 8 mg/mL concentration. The stock solution was diluted with non-cytotoxic and cytotoxic treatments in 1:1 ratio.

SB-18-4 was first dissolved in DDW containing 4% Solutol HS-15 with heating to 40° C. to 8 mg/mL concentration. The stock solution was diluted with non-cytotoxic and cytotoxic treatments in 1:1 ratio

### Treatment Groups of Experiment 1

1. 7 mice received vehicle (DDW+2% Solutol—BASF, Germany)
2. 8 mice received Hamsa treatment
3. 8 mice received Hamsa treatment in combination with SB-18 (40 mg/kg).
4. 6 mice received SB-18 in vehicle (40 mg/kg)

### Treatment Groups of Experiment 2:

1. 7 mice received vehicle (DDW+2% Solutol—BASF, Germany)
2. 8 mice received Hamsa treatment
3. 7 mice received Hamsa treatment in combination with CHAPS (40 mg/kg).
4. 7 mice received CHAPS in vehicle (40 mg/kg)

### Treatment Groups of Experiment 3:

1. 7 mice received vehicle (DDW+2% Solutol—BASF, Germany)
2. 6 mice received Hamsa treatment.
3. 7 mice received Hamsa treatment with SB-12 (2.5 mg/kg).
4. 7 mice received SB-12 in vehicle (2.5 mg/kg)

### Treatment Groups of Experiment 4:

1. 7 mice received vehicle (DDW+2% Solutol—BASF, Germany)
2. 7 mice received Hamsa treatment
3. 7 mice received Hamsa treatment with SB-12 (40 mg/kg).

### Treatment Groups of Experiment 5:

1. 6 mice received vehicle (DDW+2% Solutol—BASF, Germany)
2. 6 mice received Hamsa treatment
3. 7 mice received Hamsa treatment in combination with SB-16-4 (40 mg/kg).
4. 5 mice received SB-16-4 in vehicle (40 mg/kg)

### Treatment Groups of Experiment 6:

1. 7 mice received vehicle (DDW+2% Solutol—BASF, Germany)
2. 8 mice received Hamsa treatment
3. 7 mice received Hamsa treatment in combination with SB-18-4 (40 mg/kg).
4. 7 mice received SB-18-4 in vehicle (40 mg/kg)

Five (5) or six (6) days after inoculation, when the tumor is already palpable, the treatment was initiated by intraperitoneally (i.p.) injection of the indicated treatment, in a volume of 250 uL, once a day, every day, 6 days a week.

Tumors were measured twice a week using a “Manostat” caliber and plotted in a graph. The formula used for assessing the 3 dimensional size of the tumor was: length×width×width×0.52. The width measurement was also used as an indication for tumor height, and the 0.52 is a normalizing factor. Tumor volume was thus calculated according to the following formula: V(t)=0.52×Length×Width² (Brem H. and Folkman J. J. Pediat. Surg. 28: 445-451 (1993)). The animals were weighed once a week.

Mice receiving Hamsa treatment received a cytotoxic treatment (all four compounds of Hamsa) twice a week. On the other days, these mice were administered Cimetidine and SSZ only (see Table 1).

Mice receiving SB-12, CHAPS, SB-18, SB-16-4 or SB-18-4 received this agent daily.

### Results:

### Experiment 1: the Effect of SB-18 (40 Mg/Kg) on Tumor Growth

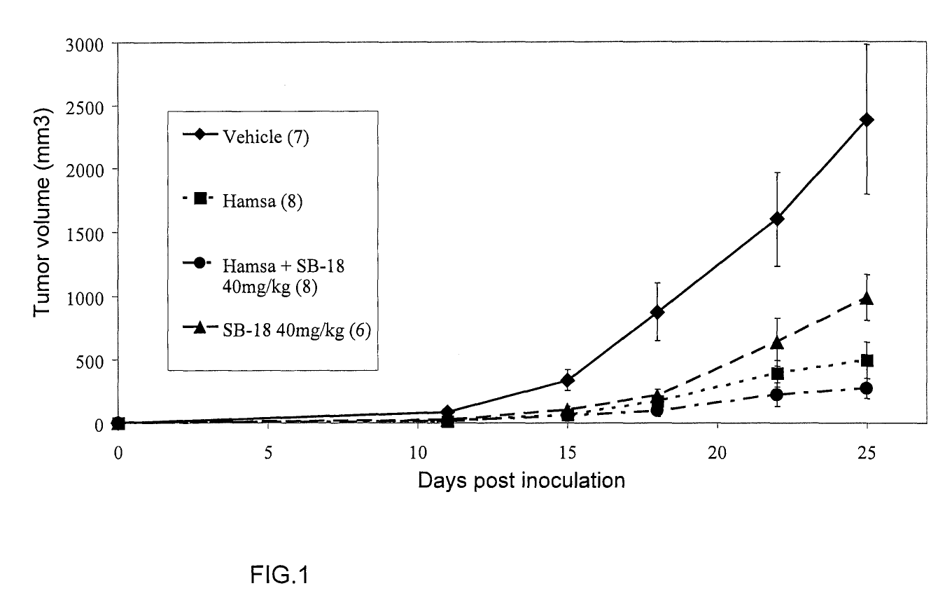

The results are depicted in FIG. 1 which demonstrates that (a) SB-18 alone has an anti-tumor effect; and (b) SB-18 has a synergistic effect in combination with Hamsa treatment on inhibition of tumor growth.

### Experiment 2: the Effect of CHAPS (40 Mg/Kg) on Tumor Growth

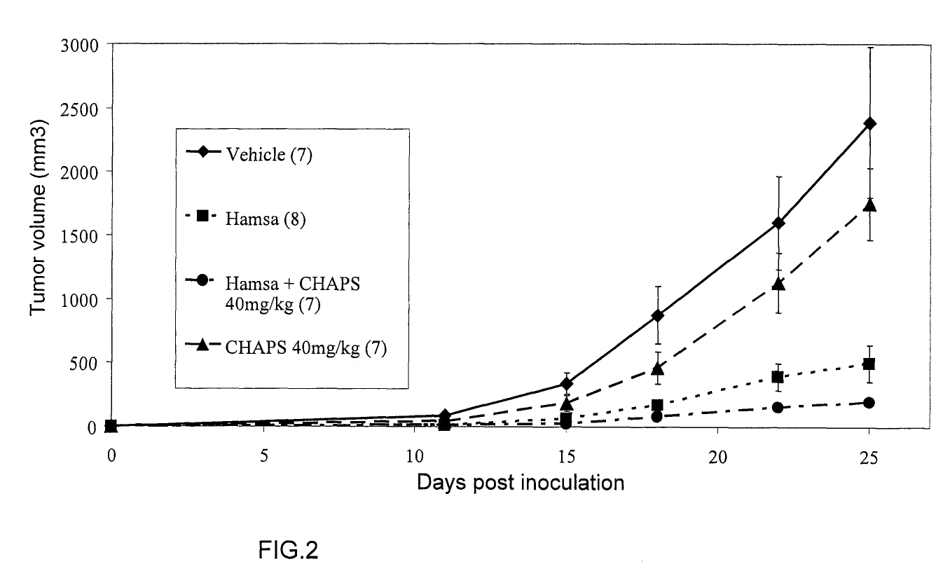

The results are depicted in FIG. 2 which demonstrates that (a) CHAPS alone has an anti-tumor effect; and (b) CHAPS has a synergistic effect in combination with Hamsa treatment on the inhibition of tumor growth.

### Experiment 3: the Effect of 2.5 Mg/Kg SB-12 on Tumor Growth

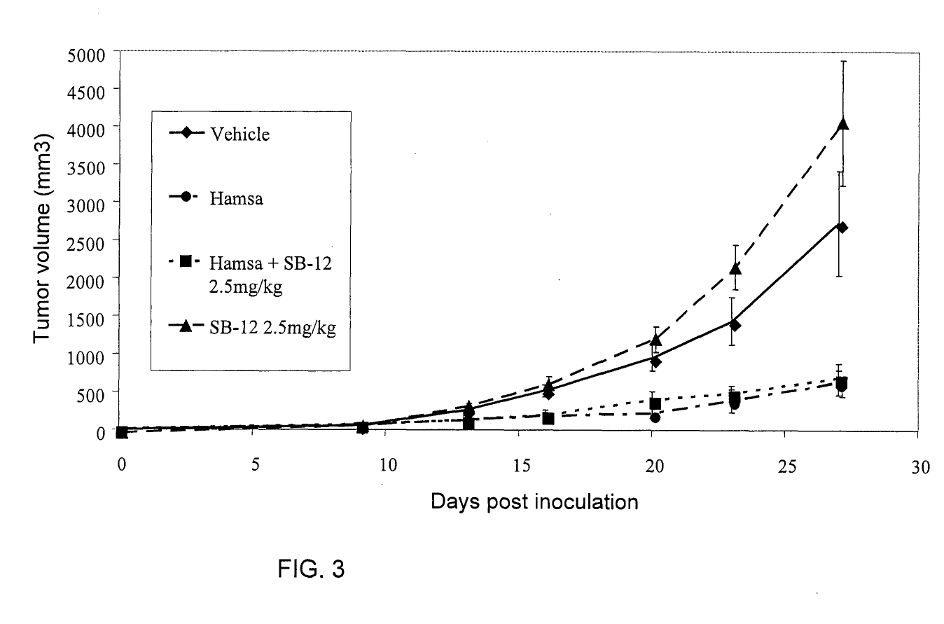

The results are depicted in FIG. 3 which demonstrate that (a) SB-12 alone does not inhibit tumor growth, but rather enhances tumor-growth; and (b) SB-12 slightly compromises the anti-tumor effect of the Hamsa treatment.

Experiment 4: the Effect of 40 Mg/Kg SB-12 in Combination with Hamsa Treatment

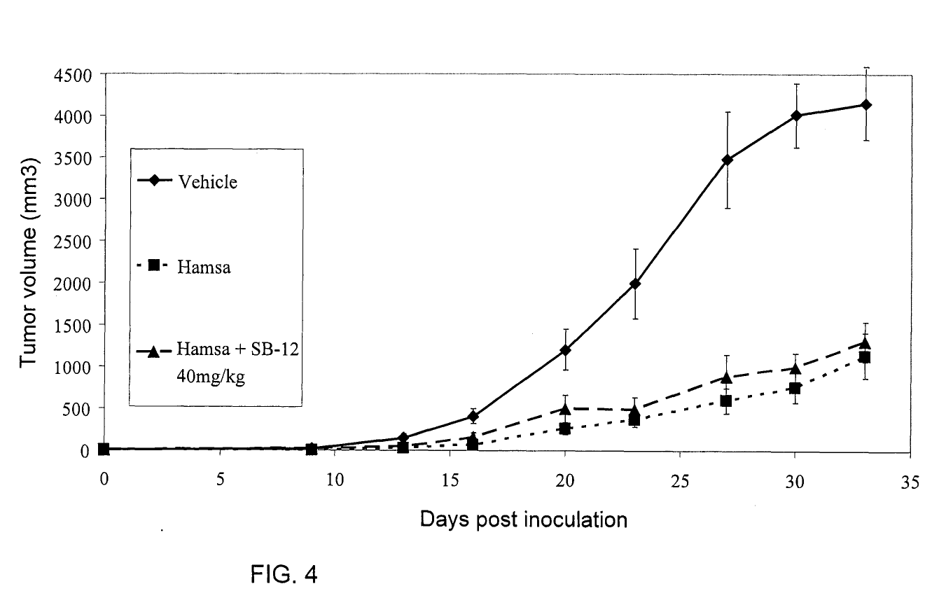

The results are depicted in FIG. 4 which demonstrate that SB-12 compromises the anti-tumor effect of Hamsa treatment also at this higher dose.

The results demonstrate that both SB-18 and CHAPS alone and in combination with Hamsa treatment inhibited tumor growth, whereas SB-12 did not.

### Experiment 5: the Effect of 40 Mg/Kg SB-16-4 on Tumor Growth

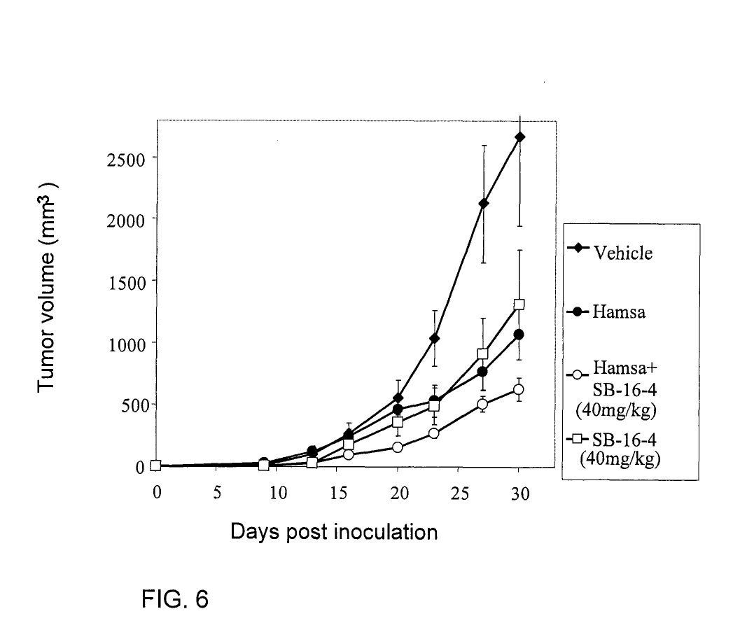

The results are depicted in FIG. 6 which demonstrates that (a) SB-16-4 alone has an anti-tumor effect; and (b) SB-16-4 has a synergistic effect in combination with Hamsa treatment on inhibition of tumor growth.

### Experiment 6: the Effect of 40 Mg/Kg SB-18-4 on Tumor Growth

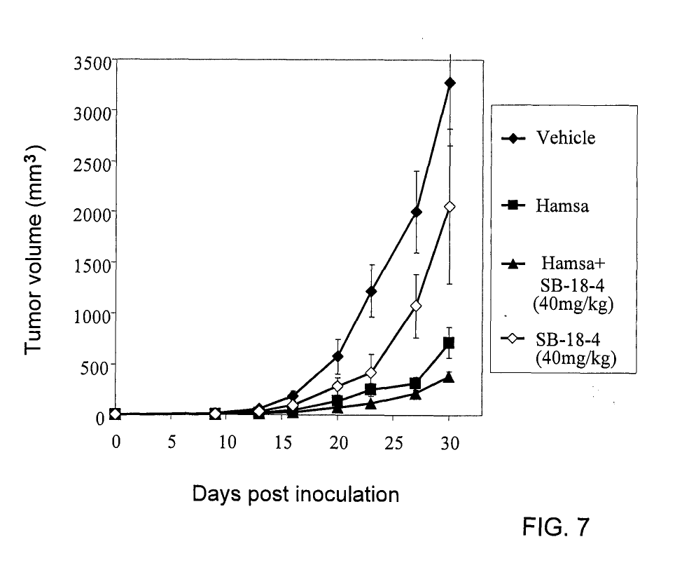

The results are depicted in FIG. 7 which demonstrates that (a) SB-18-4 alone has an anti-tumor effect; and (b) SB-18-4 has a synergistic effect in combination with Hamsa treatment on inhibition of tumor growth.

1-102. (canceled)

103. A pharmaceutical composition comprising a sulfobetaine of general formula (I) 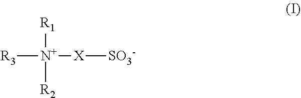
wherein
X is —(CH₂)ₙ—, wherein n is 1-4, optionally substituted by at least one group selected from —(CH₂)ₚCH₃, —(CH₂)ₚOH, —(CH₂)ₚNH₂, and —(CH₂)ₚSH; wherein p is 0-3;
R₁ and R₂ independently of each other are each selected from a group consisting of H and —(CH₂)ₘCH₃ wherein m is 0-3; or
R₁ and R₂ form together with the nitrogen atom a 5-8 membered hetero-aliphatic or hetero-aromatic ring;
R₃ is a straight or branched C₁₄-C₂₅ alkyl interrupted by at least one group selected from —C(═S)—NH—, —C(═O)—SH—, —C(═O)—O—, —NH—C(═O)NH— and —NH—C(═S)NH—, straight or branched C₁₄-C₂₅ alkenyl, straight or branched C₁₄-C₂₅ alkynyl, each of said alkenyl or alkynyl are optionally interrupted by at least one group selected from —C(═O)—NH—, —C(═S)—NH—, —C(═O)—SH—, —C(═O)—O—, —NH—C(═O)NH— and —NH—C(═S)NH—; each of said alkyl, alkenyl or alkynyl are optionally substituted with at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkylamino, dialkylamino, carboxyl, or thio; or
R₃ is a group having a formula (VIII) 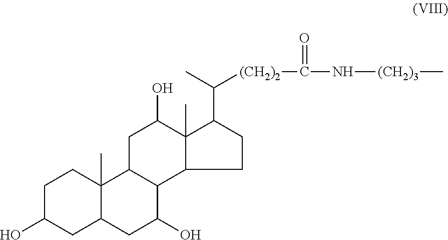
or enantiomers or diastereomers thereof; and a pharmaceutically acceptable carrier.

104. A pharmaceutical composition according to claim 103 wherein X is —(CH₂)ₙ—, wherein n is 1-4.

105. A pharmaceutical composition according to claim 104 wherein n is 3, R₁ and R₂ are CH₃.

106. A pharmaceutical composition according to claim 103 having the formula (III)

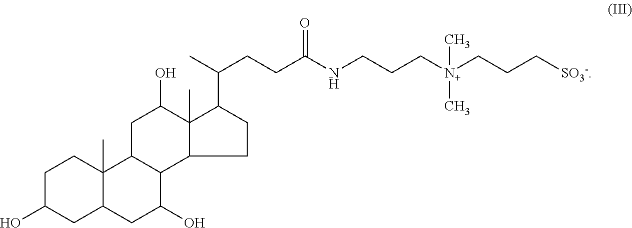

107. A pharmaceutical composition according to claim 103 having the formula (IX)

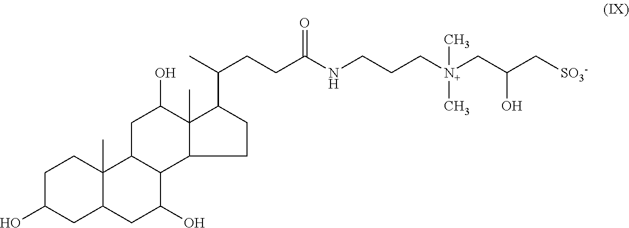

108. A pharmaceutical composition comprising a sulfobetaine of general formula (I) 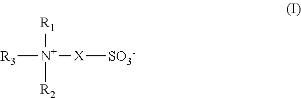
wherein
X is —(CH₂)ₙ—, wherein n is 1-4, optionally substituted by at least one group selected from —(CH₂)ₚCH₃, —(CH₂)ₚOH, —(CH₂)ₚNH₂, and —(CH₂)ₚSH; wherein p is 0-3;
R₁ and R₂ independently of each other are each selected from a group consisting of H and —(CH₂)ₘCH₃ wherein m is 0-3; or
R₁ and R₂ form together with the nitrogen atom a 5-8 membered hetero-aliphatic or hetero-aromatic ring;
R₃ is a straight or branched C₁₄-C₂₅ alkyl, straight or branched C₁₄-C₂₅ alkenyl, straight or branched C₁₄-C₂₅ alkynyl, each optionally substituted with at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkylamino, dialkylamino, carboxyl, or thio; and each optionally interrupted by at least one group selected from —C(═O)—NH—, —C(═S)—NH—, —C(═O)—SH—, —C(═O)—O—, —NH—C(═O)NH— and —NH—C(═S)NH—;
or R₃ is a group having a formula (VIII) 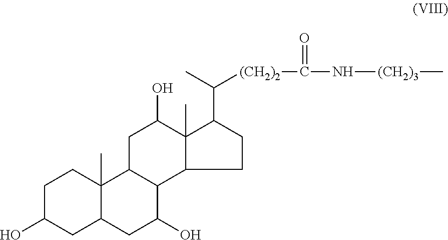
or enantiomers or diastereomers thereof and a pharmaceutically acceptable carrier; further comprising a cytotoxic agent.

109. A pharmaceutical composition according to claim 108, further comprising at least on agent selected from an anti-inflammatory agent, an NFκB inhibitor and an H2-blocker.

110. A sulfobetaine compound of formula (I) 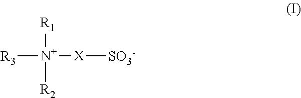
wherein
X is —(CH₂)ₙ—, wherein n is 1-4, optionally substituted by at least one group selected from —(CH₂)ₚCH₃, —(CH₂)ₚOH, —(CH₂)ₚNH₂, and —(CH₂)ₚSH wherein p is 0-3;
R₁ and R₂ independently of each other are each selected from a group consisting of H and —(CH₂)ₘCH₃ wherein m is 0-3; or
R₁ and R₂ form together with the nitrogen atom a 5-8 membered hetero-aliphatic or hetero-aromatic ring;
R₃ is a straight or branched C₁₄-C₂₅ alkyl interrupted by at least one group selected from —C(═S)—NH—, —C(═O)—SH—, —C(═O)—O—, —NH—C(═O)NH— and —NH—C(═S)NH—, straight or branched C₁₄-C₂₅ alkenyl, straight or branched C₁₄-C₂₅ alkynyl, each of said alkenyl or alkynyl are optionally interrupted by at least one group selected from —C(═O)—NH—, —C(═S)—NH—, —C(═O)—SH—, —C(═O)—O—, —NH—C(═O)NH— and —NH—C(═S)NH—; each of said alkyl, alkenyl or alkynyl are optionally substituted with at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkylamino, dialkylamino, carboxyl, or thio;
or R₃ is a group having a formula (VIII) 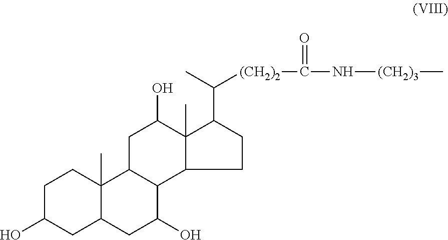
or enantiomers or diastereomers thereof and a pharmaceutically acceptable carrier for therapy.

111. A method of treating cancer comprising administering a pharmaceutical composition comprising a sulfobetaine of general formula (I) 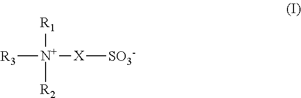
wherein
X is —(CH₂)ₙ—, wherein n is 1-4, optionally substituted by at least one group selected from —(CH₂)ₚCH₃, —(CH₂)ₚOH, —(CH₂)ₚNH₂, and —(CH₂)ₚSH; wherein p is 0-3;
R₁ and R₂ independently of each other are each selected from a group consisting of H and —(CH₂)ₘCH₃ wherein m is 0-3; or
R₁ and R₂ form together with the nitrogen atom a 5-8 membered hetero-aliphatic or hetero-aromatic ring;
R₃ is a straight or branched C₁₄-C₂₅ alkyl interrupted by at least one group selected from —C(═O)—NH—, —C(═S)—NH—, —C(═O)—SH—, —C(═O)—O—, —NH—C(═O)NH— and —NH—C(═S)NH—, straight or branched C₁₄-C₂₅ alkenyl, straight or branched C₁₄-C₂₅ alkynyl, each of said alkenyl or alkynyl are optionally interrupted by at least one group selected from —C(═O)—NH—, —C(═S)—NH—, —C(═O)—SH—, —C(═O)—O—, —NH—C(═O)NH— and —NH—C(═S)NH—; each of said alkyl, alkenyl or alkynyl are optionally substituted with at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkylamino, dialkylamino, carboxyl, or thio;
or R₃ is a group having a formula (VIII) 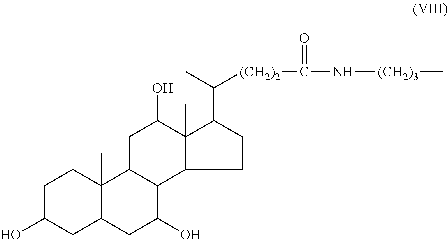
or enantiomers or diastereomers thereof; and a pharmaceutically acceptable carrier.

112. A method of treating cancer in a subject in need thereof comprising administering a pharmaceutically effective amount of a compound of formula (XVI) to the subject 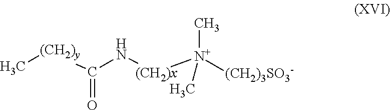
wherein x=1-4 and y=10-24.

113. A method of treating cancer in a subject in need thereof comprising administering a pharmaceutically effective amount of a compound of formula (XVII) to the subject 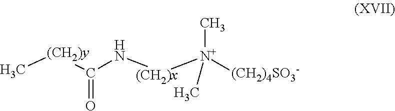
wherein x=1-4 and y=10-24.

114. A method according to claim 112 or 113, wherein x=3 and y=16.

115. A method of treating cancer in a subject in need thereof comprising administering a pharmaceutically effective amount of a compound of formula (III): 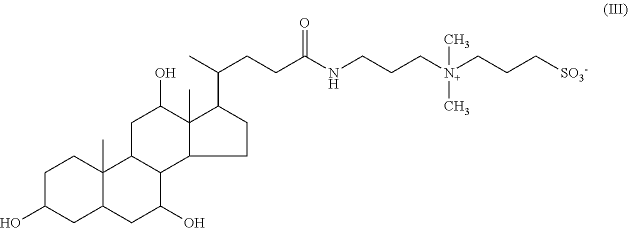
to the subject.

116. A method of treating cancer in a subject in need thereof comprising administering a pharmaceutically effective amount of a compound of formula (IX): 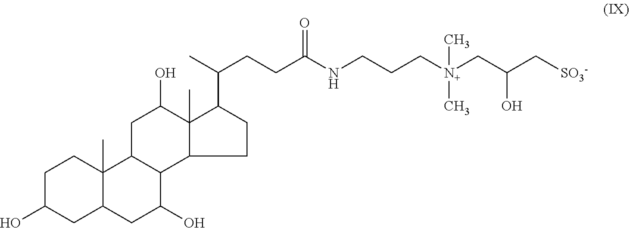
to the subject.

117. A method according to any one of claim 112, 113, 115 or 116 further comprising administration of an anti-cancer agent.

118. A method of treating cancer in a subject in need thereof comprising administering a pharmaceutically effective amount of a sulfobetaine of general formula (I), further comprising administration of an anti-cancer agent;
wherein said sulfobetaine of general formula (I) is 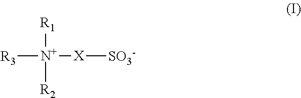
wherein
X is —(CH₂)ₙ—, wherein n is 1-4, optionally substituted by at least one group selected from —(CH₂)ₚCH₃, —(CH₂)ₚOH, —(CH₂)ₚNH₂, and —(CH₂)ₚSH; wherein p is 0-3;
R₁ and R₂ independently of each other are each selected from a group consisting of H and —(CH₂)ₘCH₃ wherein m is 0-3; or
R₁ and R₂ form together with the nitrogen atom a 5-8 membered hetero-aliphatic or hetero-aromatic ring;
R₃ is a straight or branched C₁₄-C₂₅ alkyl, straight or branched C₁₄-C₂₅ alkenyl, straight or branched C₁₄-C₂₅ alkynyl, each optionally substituted with at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkylamino, dialkylamino, carboxyl, or thio; and each optionally interrupted by at least one group selected from —C(═O)—NH—, —C(═S)—NH—, —C(═O)—SH—, —C(═O)—O—, —NH—C(═O)NH— and —NH—C(═S)NH—;
or R₃ is a group having a formula (VIII) 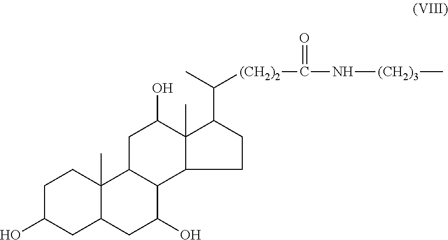
or enantiomers or diastereomers thereof and a pharmaceutically acceptable carrier.

119. A method according to claim 118, wherein said anti-cancer agent is a cytotoxic agent selected from cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, and paclitaxel or their pharmaceutically acceptable salts.

120. A method according to claim 118, further comprising administering at least one agent selected from an anti-inflammatory agent, an NFκB inhibitor and an H2-blocker.
